(19) United States
(12) Patent Application Publication
(10) Pub. No.: US 2007/0073107 A1
(43) Pub. Date: Mar. 29, 2007

(54) Methods and systems for sheathing tubular medical instruments

(75) Inventors: Kenneth A. Peartree, Redwood City, CA (US); Alexander L. Huang, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneous Systems, Inc., Mountain View, CA (US)

(21) Appl. No.: 11/233,886

(22) Filed: Sep. 23, 2005

(51) Int. Cl. A61B 1/00

(52) U.S. Cl. 600/124; 600/123; 600/121

(57) ABSTRACT

A protective sheath for endoscopes and other elongate medical devices comprises a sheath, a tissue anchor, and a stiffening element or guide. The stiffening element is used to advance the protective sheath through a working channel or other central passage of the endoscope, while the tissue anchor immobilizes a free end of the sheath at a body orifice as the endoscope is introduced. In this way, the protective sheath will drawn from the working channel, evert over a distal end of the endoscope, and remain stationary over the device relative to the body lumen as the endoscope is advanced.

### BACKGROUND OF THE INVENTION

1. Field of the Invention

The present invention relates generally to medical methods and apparatus. More particularly, the present invention relates to a protective sheath for covering endoscopes and other elongate medical devices and methods for the sheath's deployment.

Endoscopes are medical instruments for optically viewing the interior of a body lumen or hollow organ, such as the colon, bladder, stomach, or the like. The endoscopes, which may be referred to as laparoscopes, colonoscopes, gastroscopes, cystoscopes, thoracoscopes, falloposcopes, and the like, are usually introduced through a natural body orifice, and an optical imaging element at a distal end of the device is advanced to a region to be examined. The optical imaging element may be an optical waveguide which extends through the endoscope, but more recently CCD's and other electronic imaging elements have been employed. Usually, the endoscopes will incorporate a working channel through which fluid, tools, or the like, may be introduced to the body lumen or organ. Various procedures, such as biopsy, obstruction removal, polyp removal, and the like, may be performed through the working channel.

While a generally safe and relatively less invasive alternative to many open surgical procedures, the use of endoscopes is associated with certain limitations and risks. For example, the endoscope typically has a relatively large diameter in order to provide a correspondingly large working channel, and advancement of the endoscope can cause excess dilation, abrasion, and bleeding of the body lumen through which it is introduced. These injuries can, in turn, lead to infection, scarring and stricture formation. These difficulties may be exacerbated by the presence of residual chemical disinfectants used to clean the endoscopes which, for certain patients, can cause irritation. Moreover, some disinfectants are known carcinogens and contraindicated for use with cancer patients.

The use of endoscopes may also be limited in patients whose body lumens are scarred or have other strictures which may make it difficult or impossible to advance the endoscope to the target area. In such patients, forcing the endoscope past the obstruction can cause excessive pain and tissue trauma. While the use of guidewires and optionally tapered dilators can improve the chance of success, even such further measures are not always successful.

Even in successful endoscopic treatments, the need to advance the endoscope through tortuous regions while avoiding false passages can extend treatment times. In these cases, friction between the tissue and the endoscope can sometimes cause closure or “necking” of the body lumen, muscles spasms, compression of the lumen distal to the advancing endoscope, resulting in an increased risk of perforation or avulsion.

For these reasons, it would be desirable to provide improved methods and apparatus for covering and protecting endoscopes from contamination during use, as well as the patient from contamination from the endoscope. It would be further desirable if such methods and apparatus could improve or facilitate introduction of the endoscope through tortuous body lumens, particularly by reducing friction between the endoscope and the body lumen during introduction. At least some of these objectives will be met by the inventions described herein below.

2. Description of the Background Art

Sheaths for covering endoscopes and other medical devices when introduced into body lumens are described in U.S. Pat. Nos. 4,066,070; 4,741,326; 5,531,717; 5,711,841; 5,897,535; 6,240,968; and 6,447,444. Everting tubular structures for advancing medical instruments through body lumens are described in U.S. Pat. Nos. 4,321,915; 5,045,070; 5,259,364; 6,485,409; 6,699,179; and Published Application U.S. 2002/0107478.

The disclosure of the patent application has aspects in common with the disclosures of commonly owned application Ser. No. 10/794,317 (Attorney Docket No. 021807-000400US); and Ser. No. 10/794,337 (Attorney Docket No. 021807-000700US), the full disclosures of which are incorporated herein by reference.

### BRIEF SUMMARY OF THE INVENTION

The present invention provides methods and devices for loading protective sheaths over elongate medical devices which have a central passage opening at a distal end thereof. The elongate devices are most commonly endoscopes, including laparoscopes, colonoscopes, gastroscopes, thoracoscopes, cystoscopes, falloposcopes, and the like. In addition to the central passage, commonly referred to as a working channel, such endoscopes will also include an imaging element, typically a fiber optic transmission element or a CCD, at the distal end adjacent to a distal port of the working channel. Most endoscopes are also steerable using pull-wire mechanisms attached to a lever or other mechanism at a proximal hub on the endoscope.

In a first aspect of the present invention, a method for introducing an elongated medical device through a body orifice into a body lumen comprises providing an elongate device, such as an endoscope, having a central passage which opens at a distal opening (port) at its distal end, where the passage extends at least partly through the elongate device, typically extending the entire length from the distal end to the proximal end thereof. One end of the protective sheath is loaded into the central passage, typically through the distal opening in the central passage. A tissue anchor is coupled to the other end of the sheath and is positioned adjacent to the body orifice. The elongate medical device is then advanced through the body orifice and into the body lumen so that the protective sheath everts from the distal opening of the passage since the other end of the sheath is held in place by the tissue anchor. As the sheath everts, it is positioned between the exterior of the device and the interior wall of the lumen as the device is advanced. This positioning is particularly advantageous since the sheath remains stationary relative to the interior wall of the body lumen, thus helping protect the lumen against friction and convert axially forward motion into a radially outward force.

In other aspects, the elongate device will further comprise an imaging element at its distal end, typically being an endoscope such as any of the conventional endoscopes described above. In such instances, it will be desirable that the protective sheath not interfere with the ability to obtain an image through the imaging element. Thus, the protective sheath may be optically transparent, at least over a portion which will cover the imaging element after the sheath is deployed. Alternatively, the protective sheath may have an opening, e.g., be open at its distal end, so that it imaging element is uncovered after the sheath is fully deployed.

In preferred embodiments, loading comprises inserting the one end of the sheath through the distal opening of the central passage, typically using an stiffening element. The stiffening element may be a tube, typically flexible or semi-rigid, having an interior volume in which the sheath is stowed during insertion. Once the proximal end of the tube reaches a proximal port on the elongate medical device, the tube may be pulled proximally from over the sheath, leaving the sheath in place within the central passage of the device. Advantageously, co-axial tubes may be employed to provide a working channel while the sheath is within the inner lumen.

Alternatively, the stiffening element may be a rod, needle, or other hollow or solid core device which is attached to and extends from the one end of the protective sheath. Such rod-like stiffening elements may then be advanced through the working channel and drawn proximally outward through the proximal channel port on the elongate device, pulling the one end of the sheath through the working channel. As a still further alternative, the stiffening element could be used as a pusher rod which is attached to the one end of the sheath, where the one end of the sheath and the leading end of the stiffening rod are together introduced through the distal port of the working channel. The stiffening element can then be used to push the sheath proximally through the central passage until the one end of the sheath emerges from the proximal port of the channel. The sheath can then be detached from the stiffening element, and the stiffening element pulled distally to remove the element from the central passage.

The protective sheath can be composed of any one of the variety of fabrics, polymers, films, or other materials, which can be formed into tubes. Preferred are polymer tubes having a length in the range from 4 cm to 80 cm, preferably from 6 cm to 55 cm, an inner diameter in the range from 1.5 mm to 15 mm, preferably from 2 mm to 10 mm, and a wall thickness in the range from 0.01 mm to 0.05 mm. The polymer is typically a lubricious polymer film but in other cases it could be separately lubricated. Exemplary polymers include polytetrafluoroethylene (PTFE), polyethylene (PE), perfluoroalkoxy (PFA), polyurethane (PU), perfluoromethylvinylether (PFMVE), perfluoropropylvinylether (PPVE), and the like. A preferred polymer comprises tensilized PTFE/PPVE copolymer.

The methods of the present invention for introducing the elongate medical device can be used in a variety of body lumens and cavities, including the colon, the esophagus, the urethra, the ureter, the uterus, the fallopian tubes, sinus, the abdominal cavity, bronchus, duct, blood vessel, the chest cavity, joints, and the like.

In another aspect of the present invention, the protective sheath is loaded into a central lumen of an elongate medical device by providing such a device having a central passage extending at least partly therethrough. The protective sheath includes a tissue anchor at one end thereof, and the other end of the sheath is loaded into a distal opening of the central passage so that the sheath is within the passage and the tissue anchor is located near the distal end of the device. Other aspects of the methods for loading the protective sheath in the elongate medical device have been described above in the connection with the first aspect of this invention.

In a third aspect of the present invention, a protective sheath assembly for use with an elongate medical device having a central passage comprises a protective sheath and an elongate guide attachable to one end of the protective sheath. The protective sheath is adapted to cover a distal end and outer tubular surface of the device, such as an endoscope, and the elongate guide as adapted to thread or otherwise guide the sheath into and through the central passage of the device.

In preferred aspects of the apparatus, the sheath may be at least partly optically transparent so that the sheath can cover an imaging element on the elongate medical device. Alternatively, the protective sheath will have an open end which opens over the imaging element when the sheath is fully deployed from an endoscope or other medical device having an imaging element.

The elongate guide may be a tube having an interior volume for stowing the sheath. Alternatively, the sheath may be attached at one end to the elongate guide (tubular or non-tubular) so that the guide may be used to pull or push the sheath through the central lumen of the elongate medical device, generally as described above with respect to the first aspect of the present invention. In further preferred examples of the protective sheath, a tissue anchor will be coupled to the other end of the sheath, i.e. the end opposite to which the elongate guide is attached or attachable. The tissue anchor preferably comprises a ring circumscribing an opening in the sheath, where the ring is adapted to receive passage of the elongate medical devices through to allow the device to enter an orifice into a body lumen while the sheath is pulled from the central passage, everts over a distal end of the device, and covers the exterior surface of the device as it is advanced.

### BRIEF DESCRIPTION OF THE DRAWINGS

FIG. 1 illustrates three components of a protective sheath assembly constructed in accordance with the principles of the present invention.

FIG. 2 illustrates a specific embodiment of the protective sheath where the sheath is stowed in the interior of a guide tube.

FIG. 3 illustrates a second specific embodiment of the protective sheath where a guide tube is attached to a leading or distal end of the sheath to allow pulling of the sheath by the guide tube.

FIG. 4 is a detailed view taken along line 4-4 of FIG. 3 showing of the sheath as attached to a proximal end of the guide tube.

FIG. 5 illustrates a third specific embodiment of a protective sheath assembly constructed in accordance with the principles of the present invention where a distal end of the sheath is attached to a distal end of the guide tube to allow pushing of the sheath using the guide tube.

FIG. 5A is a detailed view taken along line 5A-5A of FIG. 5 showing how the sheath may be attached to the guide tube.

FIGS. 6A and 6B illustrate further specific embodiments of a protective sheath assembly constructed in accordance with the principles of the present invention where the stiffening element comprises an inner tube member.

FIG. 7 illustrates loading of the protective sheath of FIG. 2 into an endoscope.

FIG. 8 illustrates loading of the protective sheath of FIGS. 3 and 4 into an endoscope.

FIGS. 9 and 10 illustrate sequential positioning of a tissue anchor relative to a distal end of endoscope prior to introducing the endoscope into a body lumen.

FIGS. 11-13 illustrate three different protocols for introducing an endoscope through a body orifice into a body lumen according to the principles of the present invention.

### DETAILED DESCRIPTION OF THE INVENTION

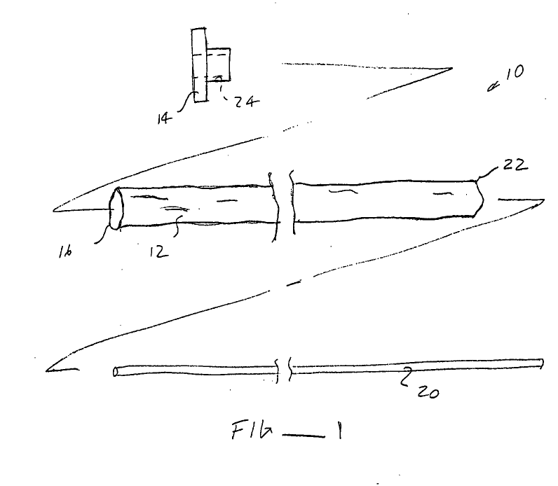

As shown in FIG. 1, protective sheath assembly 10 constructed in accordance with the principles of the present invention comprises a protective sheath 12, a tissue anchor 14 attached or attachable to an end 16 of the sheath 12, and a stiffening element or guide member 20 attachable to the other end 22 of the sheath 12. The sheath 12 will have dimensions which allow it to be deployed over the exterior of the elongate medical device, such as a conventional endoscope, and will be made from a material which allows it to be collapsed, folded, or otherwise compacted so that it may be fed into and stored within a working channel or other central passage of the elongate device. The tissue anchor 14 will typically be a ring-like structure having an opening 24 therethrough to permit advancement of the endoscope or other elongate device into a body lumen while everting the sheath 12 over the exterior of the endoscope, as will be described in more detail below.

The stiffening element or guide 20 will have dimensions which allow it to be introduced into and through a working channel or other central passage of the endoscope or other elongate medical device. It will typically have a length which is greater than that of the central passage of the elongate medical device so that it may be manually inserted through one end of the passage, pushed until it reaches and usually protrudes from the other end of the passage, and then pulled through the passage to place and/or uncover the protective sheath within the passage. The tissue anchor 14, in contrast, will remain at or near the end of the central passage through which the protective sheath had initially been introduced.

The stiffening element or guide member 20 can have a variety of different structures and may be attached to the sheath in a number of different ways. Three specific examples are illustrated in FIGS. 2-5 and 6A, 6B. In FIG. 2, the stiffening element 20 comprises a tubular structure having an interior volume which can receive and stow the sheath 12. Typically the sheath will not be fixed or otherwise attached to the stiffening element 20 so that once the stiffening element is advanced fully through the central passage of the elongate device, the stiffening element may be pulled from over the sheath 12, leaving the sheath extended in place inside the central passage. The tubular stiffening element may optionally contain an additional coaxial tube within its central passage, with the sheath stored within the annular space or lumen between the co-axial tubes (not shown). Once the outer tube is withdrawn from the central passage, the inner tube would remain in position, enclosed within the collapsed sheath. This would provide a small working lumen through the central passage, unobstructed by the undeployed sheath.

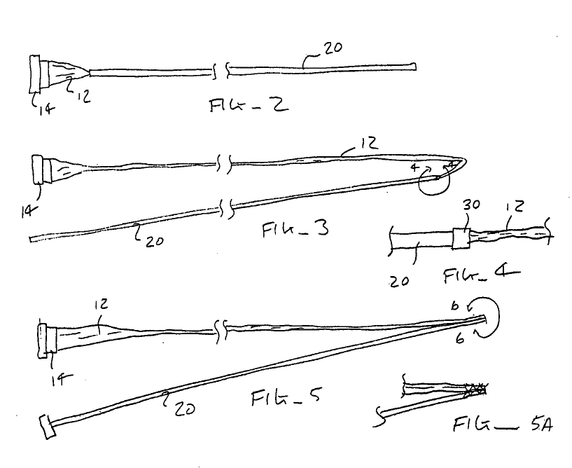

As shown in FIGS. 3 and 4, the stiffening element 20 may be a solid core rod which is removable or fixedly attached to an end of the sheath 12, for example by a coupling ring 30, as shown in FIG. 4. The stiffening element 20 is particular useful for pulling the sheath 12 through a central passage of an elongate device, as described in more detail below.

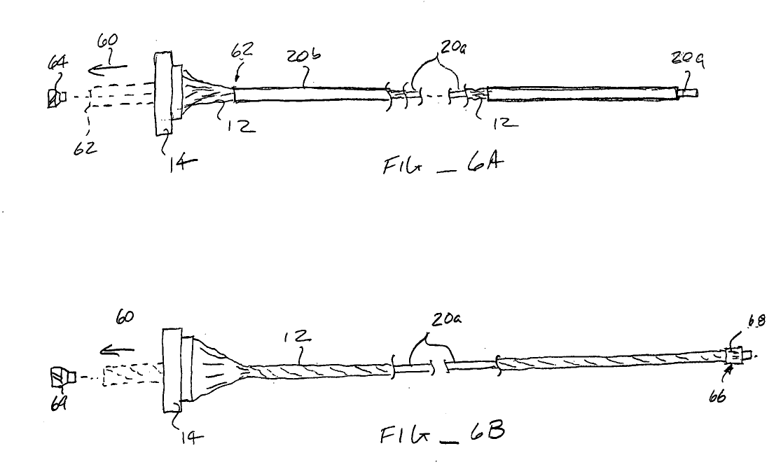

A third embodiment of a protective sheath assembly according to the present invention as shown in FIGS. 5 and 5A. There, the guide or stiffening element 20 is attached to one end of the sheath 12. As shown in FIG. 5A, the attachment has the ends of the sheath 12 and guide 20 aligned so that guide 20 is particularly suitable for pushing the sheath 12 through a central passage, as will be described in more detail below.

Still further embodiments of the protective sheath assembly of the present invention may comprise an inner tube member 20a which forms at least a portion of the stiffening member. As shown in FIG. 6A, the inner tube member 20a is used together with an outer member 20b to provide a two-component stiffening member. Protective sheath 12 and tissue anchor 14 will be generally as described for the previous embodiments. Protective sheath 12 will be held in an annular region between the outer surface of inner tube 20a and the inner surface of outer tube 20b. The assembly of the inner tube 20a, outer tube 20b, and sheath 12 may be introduced into the central passage of an endoscope or other elongate medical device by advancing the assembly in the direction of arrow 60 so that an end 62 of the assembly eventually emerges from a proximal end of the central passage, as will be described in greater detail below. Once the proximal end has emerged, the outer tube 20 may be removed, leaving the inner tube 20a in place to provide an access lumen through the interior of the sheath. Optionally, a removable luer or other fitting 64 may be provided for facilitating access to the inner lumen of the inner tube 20a.

The embodiment of FIG. 6B is similar to that of 6A, except that the outer tube 20b is eliminated. The protective sheath 12 is wrapped about the inner tube 20a and optionally secured to the inner tube at an end 66, typically using a ring or other fastener 68. The assembly is again introduced in the direction of arrow 60, and the inner tube 20a may be left in place after deployment of the sheath and introduction of the elongate device, further optionally employing a luer 64.

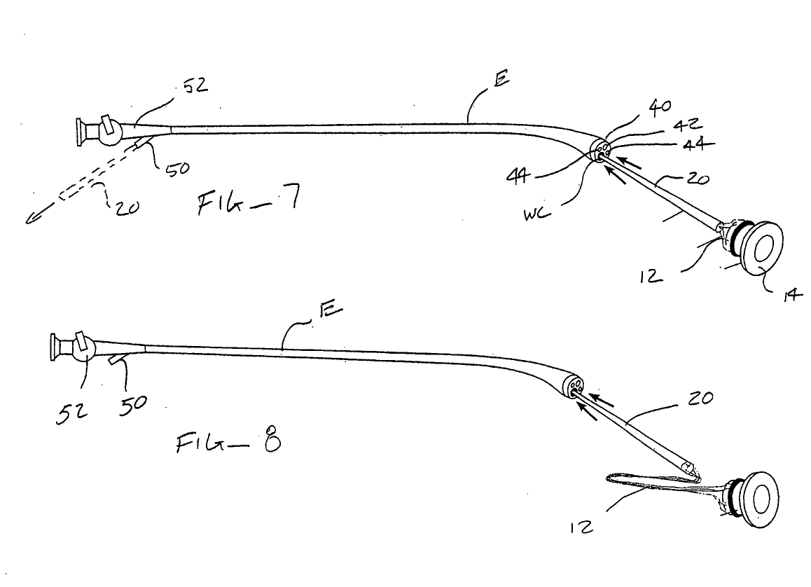

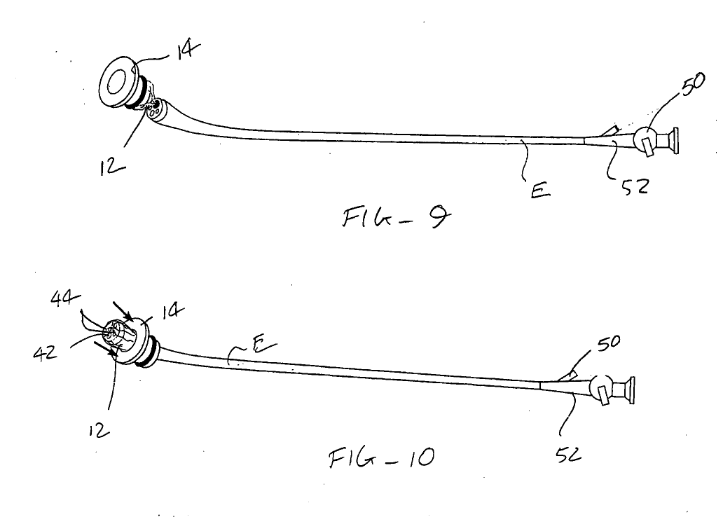

Referring now to FIG. 7, the protective sheath assembly of FIG. 2 may be introduced through the working channel WC at the distal end 40 of an endoscope E. In addition to the working channel WC, the endoscope E will usually include an optical imaging element 42 and a pair of light transmission elements 44. The tubular stiffening element 20 is advanced through the working channel WC of the endoscope E until a leading end of the stiffening element 20 emerges from the port 50 on the working hub 52 of the endoscope. The leading end is shown in a broken line in FIG. 7, and may be pulled completely from the endoscope, leaving the protective sheath 20 within the working channel WC and the tissue anchor 14 at the distal end of the endoscope E, as shown in FIG. 9.

Alternatively, the protective sheath assembly of FIGS. 3 and 4 may be introduced to the endoscope E, as shown in FIG. 8. The guide 20 is introduced so that its distal end is exposed from the working channel WC and the distal end is pulled by the sheath 12 therethrough. (Note that sheath 12 will usually be longer than illustrated to accommodate the endoscope shown.) After the guide 20 has been pulled completely from the port 50, it may optionally be detached from the sheath, leaving the sheath in place in the working channel WC. If the guide tube is removed, the sheath 12 will be in place within the endoscope E, also as shown in FIG. 9 herein.

To prepare endoscope E having the sheath assembly thereon for use, the tissue anchor 14 will be pushed back over the distal end of the endoscope E to begin everting of the sheath 12, as shown in FIG. 10. The sheath will cover the imaging element 42 and the light transmission members 44. In such instances, it is desirable that at least the portion of the sheath 12 which covers these elements be optically transparent in order to permit imaging in an otherwise conventional manner during advancement of the endoscope E.

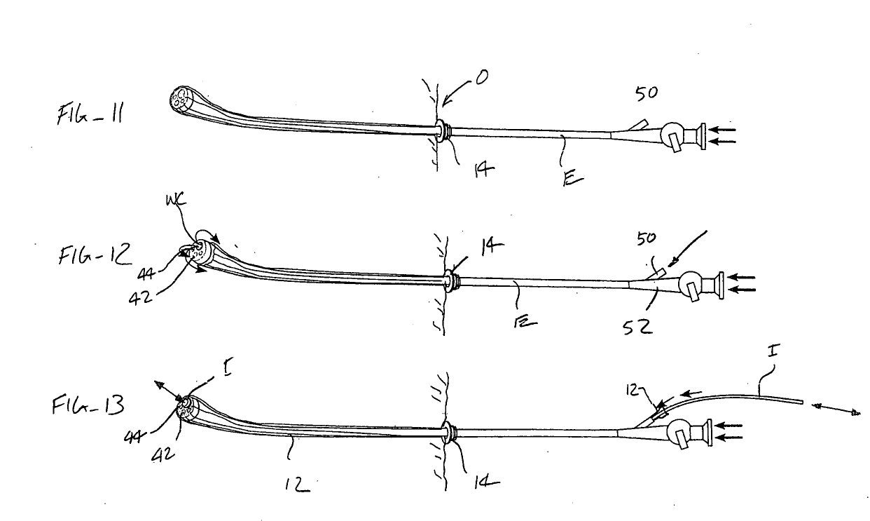

Referring now to FIG. 11, the endoscope E as shown in FIG. 10, may be advanced into a body lumen by first engaging the tissue anchor 14 at the orifice O. The leading end of the endoscope E is then advanced into the body lumen, drawing the protective sheath out from the central passage of the endoscope E as the endoscope E is advanced. The sheath 12 will thus continue to evert around the distal end of the endoscope E but will remain essentially stationary relative to the wall of the body lumen, thus providing protection to the body lumen and generally facilitating advancement of the endoscope E.

As shown in FIG. 12, in some embodiments of the present invention, the endoscope E may be advanced fully until the sheath 12 opens up over the distal end of the endoscope E, thus exposing the working channel WC as well as the optical element 42 and light transmission 44. In this way, the endoscope E can be delivered altraumatically and then used in an entirely conventional manner without any restriction or any interference from the protective sheath 12.

Referring now to FIG. 13, the endoscope E as illustrated in FIG. 11 can be used to introduce a working tool or implement by introducing the implement I through at least a portion of the interior of the sheath which remains in the working channel WC. In that instance, a distal end of the implement I will emerge from the leading end of the endoscope E and will not be covered by the protective sheath. The imaging element 42 and light transmission element 44, in contrast, will remain within the protective sheath 12. As described above, however, the protective sheath 12 is preferably optically transparent allowing the imaging system to function while the working implement I will have direct access to the tissue of the body lumen or cavity.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for introducing an elongated medical device through a body orifice into a body lumen, said method comprising:
providing an elongate device having a central passage extending at least partly therethrough with an opening at a distal end thereof;
loading one end of a protective sheath in the central passage;
positioning a tissue anchor coupled to the other end of the sheath adjacent to the body orifice; and
advancing the elongate medical device through the tissue anchor and body orifice and into the body lumen so that the protective sheath everts from the passage and is positioned between the exterior of the device and interior wall of the lumen as the device is advanced.

2. A method as in claim 1, wherein the elongate device comprises an imaging element at the distal end of the device.

3. A method as in claim 2, wherein the elongate device is an endoscope.

4. A method as in claim 2, wherein an optically transparent portion of the protective sheath is aligned over the imaging element when the elongate device has been advanced to a target location in the body lumen.

5. A method as in claim 2, wherein the protective sheath is open over the imaging element when the elongate device has been advanced to a target location in the body lumen.

6. A method as in claim 1, wherein loading comprises inserting the one end of the sheath through the distal opening of the central passage.

7. A method as in claim 6, wherein the one end of the sheath is inserted using a stiffening element.

8. A method as in claim 7, wherein the stiffening element is a tube having an interior volume which carries the sheath as the tube is inserted within the central passage.

9. A method as in claim 7, wherein the stiffening element is a rod or a tube attached to the one end of the sheath, wherein the rod or tube is inserted through the central passage of the elongate device in advance of the sheath, and wherein a lumen of the tube may remain in place to provide access through the central passage.

10. A method as in claim 9, wherein the rod is inserted through the central passage of the elongate device in advance of the sheath.

11. A method as in claim 9 wherein a leading end of the rod is attached to the one end of the sheath and the rod pushes the one end of the sheath toward a proximal end of the device.

12. A method as in claim 1, wherein the protective sheath comprises a polymeric tube.

13. A method as in claim 12, wherein the polymeric tube has a length in the range from 4 cm to 80 cm, an inner diameter in the range from 1.5 mm to 15 mm, and a wall thickness in the range from 0.01 mm to 0.05 mm.

14. A method as in claim 12, wherein the polymer is a lubricious polymer.

15. A method as in claim 12, wherein the polymer is lubricated.

16. A method as in claim 12, wherein the polymer is selected from the group consisting of polytetrafluoroethylene (PTFE), polyethylene (PE), perfluoroalkoxy (PFA), polyurethane (PU), perfluoromethylvinylether (PFMVE), perfluoropropylvinylether (PPVE).

17. A method as in claim 16, wherein the polymer comprises tensilized PTFE/PPE copolymer.

18. A method as in claim 1, wherein the body lumen is selected from the group consisting of a colon, esophagus, urethra, ureter, uterus, fallopian tube, sinus, abdominal cavity, bronchus, duct, blood vessel, chest cavity, and joints.

19. A method for loading a protective sheath in a central passage of an elongate medical device, said method comprising:
providing an elongate medical device having a central passage extending at least partly therethrough;
providing a protective sheath having a tissue anchor at one end thereof; and
loading the other end of the protective sheath into a distal opening of the central passage so that sheath is within the passage and the tissue anchor is near the distal end of the device.

20. A method as in claim 19, wherein the elongate device comprises an imaging element at the distal end of the device.

21. A method as in claim 20, wherein the elongate device is an endoscope.

22. A method as in claim 20, wherein an optically transparent portion of the protective sheath is aligned over the imaging element when the elongate device has been advanced to a target location in the body lumen.

23. A method as in claim 20, wherein the protective sheath is open over the imaging element when the elongate device has been advanced to a target location in the body lumen.

24. A method as in claim 19, wherein loading comprises inserting the one end of the sheath through the distal opening of the control passage.

25. A method as in claim 24, wherein the one end of the sheath is inserted using a stiffening element.

26. A method as in claim 25, wherein the stiffening element is a tube having an interior volume which carries the sheath as the tube is inserted within the central passage.

27. A method as in claim 25, wherein the stiffening element is a rod or tube attached to the one end of the sheath, wherein the rod is inserted through the central passage of the elongate device in advance of the sheath, and wherein a lumen of the tube may remain in place to provide access through the central passage.

28. A method as in claim 27, wherein the rod is inserted through the central passage of the elongate device in advance of the sheath.

29. A method as in claim 27, wherein a leading end of the rod is attached to the one end of the sheath and the rod pushes the one end of the sheath toward a proximal end of the device.

30. A method as in claim 19, wherein the protective sheath comprises a polymeric tube.

31. A method as in claim 30, wherein the polymeric tube has a length in the range from 4 cm to 80 cm, an inner diameter in the range from 1.5 mm to 15 mm, and a wall thickness in the range from 0.01 mm to 0.05 mm.

32. A method as in claim 30, wherein the polymer is a lubricious polymer.

33. A method as in claim 30, wherein the polymer is lubricated.

34. A method as in claim 30, wherein the polymer is selected from the group consisting of polytetrafluoroethylene (PTFE), polyethylene (PE), perfluoroalkoxy (PFA), polyurethane (PU), perfluoromethylvinylether (PFMVE), perfluoropropylvinylether (PPVE).

35. A method as in claim 34, wherein the polymer comprises tensilized PTFE/PPVE copolymer.

36. A protective sheath assembly for use with an elongate medical device having a central passage extending at least partly therethrough, said assembly comprising:
a protective sheath for covering a distal end and outer tubular surface of the device; and
an elongate guide attachable to one end of the protective sheath for threading the sheath into the central passage.

37. A protective sheath assembly as in claim 36, wherein the protective sheath is at least partly optically transparent so that the sheath can be covered over a medical device having an imaging element.

38. A protective sheath assembly as in claim 36, wherein the protective sheath has an open end which receives the imaging element where the sheath is covered over a medical device having the imaging element.

39. A protective sheath assembly as in claim 36, wherein the protective sheath comprises a polymeric tube.

40. A protective sheath assembly as in claim 39, wherein the polymeric tube has a length in the range from 4 cm to 80 cm, an inner diameter in the range from 1.5 mm to 15 mm, and a wall thickness in the range from 0.01 mm to 0.05 mm.

41. A protective sheath assembly as in claim 39, wherein the polymer is a lubricious polymer.

42. A protective sheath assembly as in claim 39, wherein the polymer is lubricated.

43. A protective sheath assembly as in claim 39, wherein the polymer is selected from the group consisting of polytetrafluoroethylene (PTFE), polyethylene (PE), perfluoroalkoxy (PFA), polyurethane (PU), perfluoromethylvinylether (PFMVE), perfluoropropylvinylether (PPVE).

44. A protective sheath assembly as in claim 43, wherein the polymer comprises tensilized PTFE/PPVE copolymer.

45. A protective sheath assembly as in claim 36, wherein the elongate guide comprises a tube having an interior volume for stowing the sheath.

46. A protective sheath assembly as in claim 36, wherein the elongate guide comprises a rod attached at one end to the sheath.

47. A protective sheath assembly as in claim 46, wherein the rod is arranged to pull the sheath through the central passage of the medical device.

48. A protective sheath assembly as in claim 46, wherein the rod is arranged to push the sheath through the central passage.

49. A protective sheath assembly as in claim 36, wherein the elongate guide comprises a tube adapted to be positioned within the sheath while the sheath is being threaded.

50. A protective sheath assembly as in claim 36, further comprising a tissue anchor coupled to the other end of the sheath.

51. A protective sheath assembly as in claim 50, wherein the tissue anchor comprises a ring circumscribing the other end of the sheath, an opening of the ring being adopted to receive passage of the elongate medical device therethrough to effect eversion of the sheath over the device.

52. A protective sheath assembly as in claim 36, wherein the elongate guide comprises an inner tube and an outer tube, wherein the protective sheath is carried in an annular space between the two tubes while the sheath is being threaded.
